(19) United States
(12) Patent Application Publication
(10) Pub. No.: US 2012/0022262 A1
(43) Pub. Date: Jan. 26, 2012

(54) PROCESS OF HYDROLYSIS OF HETEROAROMATIC NITRILS IN AQUEOUS FLUIDS

(76) Inventors: Lothar Ott, Visp (CH); Andreas Heyl, Termen (CH); Norbert Clausen, Veyras (CH); Gregor Michalik, Darmstadt (DE); Herbert Vogel, Nauheim (DE)

(21) Appl. No.: 12/529,757

(22) PCT Filed: Apr. 21, 2008
(86) PCT No.: PCT/EP2008/003186

§ 371 Date: Aug. 15, 2011

(30) Foreign Application Priority Data
Apr. 20, 2007 (EP) 07008087.4
May 4, 2007 (EP) 07009045.1

(51) Int. Cl. C07D 211/72; C07D 213/803

(52) U.S. Cl. 546/317; 546/319

(57) ABSTRACT

The present invention relates to a process for the hydrolysis of nitriles of the formulae

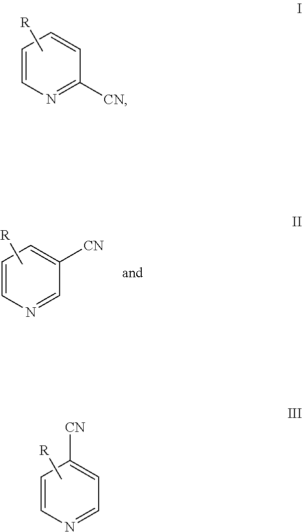

and respectively of amides of the formulae

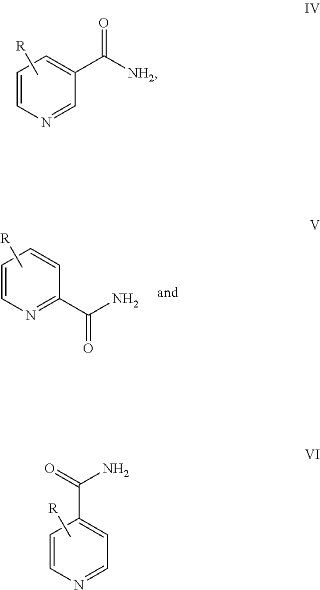

in which R is in each case hydrogen or C₁₋₂₀-alkyl, to give the corresponding amides and/or acids and respectively to give the corresponding acids.

The present invention relates to a process for the hydrolysis of compounds of the formulae

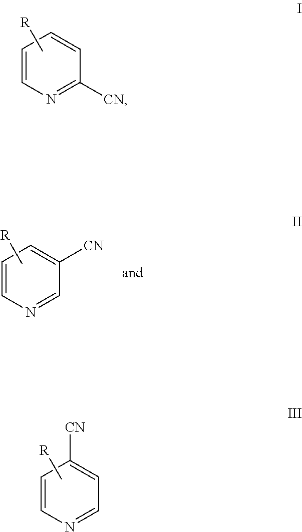

in which R is hydrogen or C₁₋₂₀-alkyl, in aqueous fluids under high pressure conditions.

Heteroaromatic nitriles of the formula I or II, in particular nicotinonitrile (NN), are important intermediates, inter alia, in the preparation of “nicotinates”, such as nicotinamide (NAM) and nicotinic acid (NAC). NAM and NAC are, for example, added to foods as vitamins or used as building blocks in the preparation of pharmaceutically active compounds.

It is known that compounds of the formulae I to III can be hydrolysed stepwise via the acid amides of the formulae

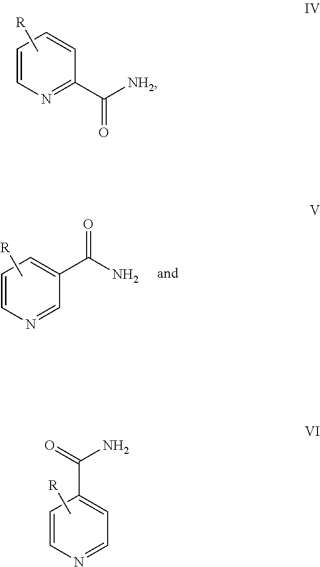

in which R is as defined above, to give the acids of the formulae

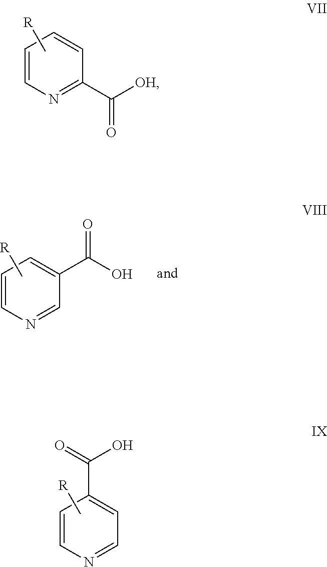

in which R is as defined above.

However, in the known processes for the preparation of NAM and NAC from NN, high salt loads are produced in the production cycle and accordingly are increasingly meeting with acceptance problems. For some time, demands have been growing to reduce the salt load in production cycles since the disposal of such wastewater, for example by incineration or dumping, is becoming increasingly expensive. Furthermore, the salts can only be removed at great expense from the products likewise partially present in salt form. This salt load is especially problematic with NAM and NAC if these are used as food supplements or animal feed supplements. The rate of hydrolysis increases strongly at relatively high temperatures but NAM and NAC decompose at relatively high temperatures and form the corresponding pyridines which, due to a negative odorous note, limit the use of the acids and amides produced and in the animal feed industry.

According to U.S. Pat. No. 5,756,750, the hydrolysis of NN can terminate, at a low sodium hydroxide concentration (14 parts of base to 100 parts of NN), in NAM and, at an equimolar sodium hydroxide concentration, in NAC. After neutralization with HCl, approximately one part of NaCl per 2 parts of NAC is obtained as waste product. An additional possibility for the preparation of NAC from NN is enzymatic hydrolysis. The nitrilases used in this connection require for the most part complex media compositions. Furthermore, these enzymes produce low space-time yields and require salting out of the NAC with an amount of salt resulting therefrom (Process Biochemistry, 2006, 41(9), 2078-2081, and JP 2005176639). Furthermore, in the enzymatic process, a loss in activity of the enzymes can often be detected (Journal of Molecular Catalysis B: Enzymatic, 2006, 39(1-4), 55-58).

It was accordingly an object of the present invention to develop a process which is characterized by a marked reduction in the salt loads with high space-time yields. Furthermore, the product should be largely free from pyridine derivatives.

This object was achieved according to claim 1.

A process is claimed for the hydrolysis of compounds of the formulae

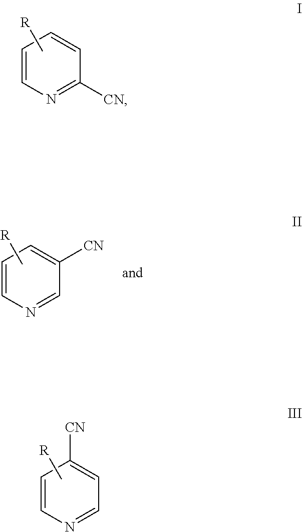

in which R is hydrogen or C₁₋₂₀-alkyl,
to give the corresponding amides and/or acids in aqueous fluids, at a temperature of at least 100° C., preferably of at least 150° C., particularly preferably of at least 200° C., and a pressure of at least 10 MPa, preferably of at least 15 MPa, particularly preferably of at least 20 MPa, if appropriate in the presence of an acid or basic catalyst.

Depending on the way the reaction is carried out, mixtures with different compositions are obtained.

The first stage, the hydrolysis of the nitriles, takes place, under the conditions according to the invention, faster than the second stage, the hydrolysis of the amides. With suitable reaction control, the hydrolysis can be stopped predominantly at the stage of the amides of the formulae IV to VI or can be continued up to the next stage of the corresponding acids of the formulae VII to IX. In the examples referred to below, it is shown that the product composition can be controlled over a wide range by the choice of suitable parameters (temperature, pressure, type and amount of catalyst). This is particularly advantageous for the compounds NAM and NAC, which can also be used as mixtures in the food and animal feed field, and with which the product, after removal of the solvents and catalysts optionally used, can be directly reused.

In the process according to the invention for the preferred preparation of compounds of the formulae IV to IX, a temperature of at most 280° C., preferably of at most 270° C., particularly preferably of at most 250° C., is advisably not exceeded. Above 400° C., the acids of the formulae VII to IX are almost quantitatively decarboxylated to give the corresponding pyridine derivatives.

In a preferred process variant, the reaction is carried out continuously in a flow reactor with at least one reaction region.

Suitable reactors are, for example, flow reactors, with or without static mixers, or pressure-resistant microreactors, with or without separate mixing regions. When homogeneous mixtures are introduced, special mixing devices can be dispensed with.

When starting materials and aqueous phase are introduced separately, a mixing device is advantageous. Among the microreactors, metal-based ones, which can be quickly and precisely adjusted to the correct temperature, are suitable in particular. If appropriate, suitable flow reactors comprise additional inlets for the charging of catalysts along the reaction region. This additional charging is advantageous in particular for the introduction of gaseous ammonia when the reaction is carried out under basic conditions.

Suitable flow reactors exhibit, if appropriate, one or more subsequent reaction regions.

In a static mixer, the residence time in the reaction region is preferably between 0.1 and 7000 seconds, particularly preferably between 30 and 6000 seconds.

The term “aqueous fluids” is to be understood as meaning, in the present process, water-comprising solvents, in particular aqueous liquids, in which the other solvent constituents are inert with regard to the starting compounds and the products and are not decomposed under the reaction conditions. Suitable solvent constituents are, in addition to water, also organic compounds, such as C₁₋₁₀-alcohols, C₁₋₁₀-carboxylic acids, esters of the abovementioned alcohols and carboxylic acids, methyl tert-butyl ketone and paraffins. The carboxylic acids mentioned can in this connection, in particular at the high reaction temperatures, act simultaneously as solvent and as catalyst. Preferably, the fluid is composed predominantly of water.

In a preferred process variant, the aqueous fluid at the beginning of the reaction comprises a nitrile of the formula I, II or III in an amount of 0.05 to 30% by weight, particularly preferably of 10 to 20% by weight.

In an additional preferred variant, the process is carried out at a pressure in the range from at least 10 MPa, preferably from 20 to 60 MPa, particularly preferably from 20 to 35 MPa.

Generally, with increasing temperature or with increasing residence time in the reactor, the formation of the acids of the formulae VII to IX predominates over the formation of the amides of the formulae IV to VI. If the process is carried out in an aqueous fluid with a pH which has not been preset, thus without addition of acids or bases, the product ratio of the acids to the amides can be controlled within a wide range by temperature and pressure. However, the reaction times under these conditions are relatively long and favour the decarboxylation of the nicotinic acid formed to give the corresponding pyridine derivatives.

Basic or acid catalysts can also, in the process according to the invention, advantageously be added in order to increase the reaction rate and for additional control of the selectivity. Basic and acid catalysts can also reduce the formation of byproducts. Both acids and bases which are at least partially soluble in the reaction mixture and acids and bases which are heterogeneous are suitable as catalysts.

When acid catalysts are used, in addition to a general increase in the reaction rate, the formation of undesirable decarboxylated pyridines is favoured.

When basic catalysts are used, in addition to a general increase in the reaction rate, the selectivity with regard to the formation of the acids of the formulae VII to IX is improved. At comparable temperatures and reaction times, the use of basic catalysts has, in comparison with the process using acid catalysts or in the absence of catalysts, the additional advantage of a reduced decarboxylation.

The catalyst is particularly preferably used in an amount of at least 0.3 mol/l.

In a process variant, the catalyst is an acid catalyst.

Suitable acid catalysts are in particular low-molecular-weight organic acids, such as, for example, linear or branched C₁₋₆-carboxylic acids, or inorganic acids, such as, for example, oxidizing or nonoxidizing Brönsted acids or Lewis acids, but also inorganic solid acids, such as poly acids and heteropoly acids.

The inorganic acids are preferably chosen from the group consisting of hydrohalic acids, sulphuric acid, sulphurous acid, phosphoric acid, phosphorous acid, Lewis acids, polyphosphoric acids, polytungstic acids and mixtures thereof.

In an alternative process variant, the catalyst is a basic catalyst, in particular an organic or inorganic base.

A suitable basic catalyst is in particular an inorganic base chosen from the group consisting of hydroxides and oxides of metals of the first and second main groups. Ammonia, triethylamine, trimethylamine and mixtures of the abovementioned bases are also suitable. Alkali metal and alkaline earth metal hydroxides, alkali metal oxides and ammonia are suitable in particular.

A catalytic action already occurs, in the case of basic catalysts, in concentrations from 100 ppm (g/g), based on the starting material. In a preferred embodiment, with basic catalysis, the pH is >7. Although basic catalysts can be used in any concentration at all, a concentration of at least 0.3 mol/l is also advantageous here. At concentrations in particular of less than 0.3 mol/l, the tendency towards decarboxylation of the NAC to give pyridine derivatives increases.

Since the reaction takes place under pressure, the possibility exists of adding gaseous bases, such as ammonia or trimethylamine, under pressure. Particularly preferably, ammonia is used as gaseous base. In a very particularly preferred embodiment, gaseous ammonia is additionally added under pressure in the presence of a basic catalyst. Ammonia is particularly advantageous as basic catalyst since ammonia can be easily removed from the product, for example under vacuum, and accordingly does not require any neutralization.

In the two-stage hydrolysis of nitriles via the corresponding acid amides to give the acids, the second stage is rate-determining. As shown by Examples 1 to 4, the nitrile concentration, in particular in the presence of a catalyst, decreases markedly faster than the acid concentration increases. Due to the difference in the hydrolysis rates, the ratio of the amides to the acids can, depending on the reaction parameters, be controlled within a broad range. The hydrolysis of the nitriles to give the amides proceeds in this connection to completion and, under the reaction conditions, irreversibly, while the hydrolysis of the amides to give the corresponding acids, in particular when using ammonia as basic catalyst, is subject to a reaction equilibrium. Furthermore, the hydrolysis of the amides can also be carried out under the conditions according to the invention if the amides have been synthesized in another way or the nitriles have been hydrolysed separately in another way. The amides can, for example, be obtained chemically, even at standard pressure and standard temperature, from the nitriles by hydrolysis. However, the amides can also be obtained biotechnologically by enzymatic hydrolysis of the nitriles, as disclosed, for example, in WO-A 99/05306.

The first stage in the process according to the invention, thus the hydrolysis of the nitriles to give the amides, can also be carried out without the use of pressure or without additional applied pressure.

An additional aspect of the present invention is accordingly the hydrolysis of amides of the formulae

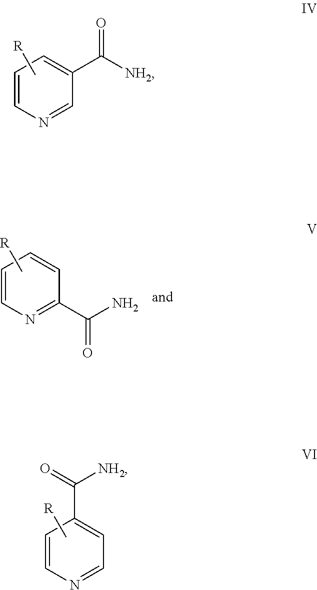

in which R is hydrogen or C₁₋₂₀-alkyl, to give the corresponding acids, at a temperature of at least 100° C., preferably of at least 150° C., particularly preferably of at least 200° C., and a pressure of at least 10 MPa, preferably of at least 15 MPa, particularly preferably of at least 20 MPa, if appropriate in the presence of an acid or basic catalyst.

A temperature of 280° C., preferably of 270° C., particularly preferably of 250° C., is not advisably exceeded for the hydrolysis of the amides.

In a preferred process variant, the reaction is carried out continuously in a flow reactor. Suitable reactors are, for example, flow reactors, with or without static mixers, or pressure-resistant microreactors, with or without separate mixing regions. When homogeneous mixtures are introduced, special mixing devices can be dispensed with. When starting materials and aqueous phase are introduced separately, a mixing device is advantageous. Among the microreactors, metal-based ones, which can be quickly and precisely adjusted to the correct temperature, are suitable in particular. If appropriate, suitable flow reactors comprise additional inlets for the charging of catalysts along the reaction region. This additional charging is advantageous in particular for the introduction of gaseous ammonia when the reaction is carried out under basic conditions.

Suitable flow reactors exhibit at least one reaction region and, if appropriate, one or more subsequent reaction regions.

In a static mixer, the residence time in the reaction region is preferably between 10 and 7000 seconds, particularly preferably between 30 and 6000 seconds.

The term “aqueous fluids” is to be understood as meaning, in the present process, water-comprising solvents, in particular aqueous liquids, in which the other solvent constituents are inert with regard to the starting compounds and the products and are not decomposed under the reaction conditions. Suitable solvent constituents are, in addition to water, also organic compounds, such as C₁₋₁₀-alcohols, C₁₋₁₀-carboxylic acids, esters of the abovementioned alcohols and carboxylic acids, methyl tert-butyl ketone and paraffins. The carboxylic acids mentioned can in this connection, in particular at the high reaction temperatures, act simultaneously as solvent and as catalyst. Preferably, the fluid is composed predominantly of water.

In a preferred process variant, the aqueous fluid at the beginning of the reaction comprises an amide of the formula IV, V or VI in an amount of 0.05 to 30% by weight, particularly preferably of 10 to 20% by weight.

In an additional preferred variant, the process is carried out at a pressure in the range from at least 10 to at most 60 MPa, preferably from 20 to 60 MPa, particularly preferably from 20 to 35 MPa.

Basic or acid catalysts can also, in the process according to the invention, advantageously be added in order to increase the reaction rate and for control of the selectivity. Basic and acid catalysts can also reduce the formation of byproducts. Both acids and bases which are at least partially soluble in the reaction mixture and acids and bases which are heterogeneous are suitable as catalysts.

The use of acid catalysts results, in comparison with a catalyst-free process, in a general increase in the reaction rate. However, the formation of decarboxylated pyridines, undesirable per se, is also possibly favoured.

The use of basic catalysts likewise causes a general increase in the reaction rate. At comparable temperatures and reaction times, the use of basic catalysts has, in comparison with the process using acid catalysts or in the absence of catalysts, the additional advantage of a reduced decarboxylation.

The catalyst is particularly preferably used in an amount of at least 0.3 mol/1.

In a particularly preferred process variant, the catalyst is an acid catalyst.

Suitable acid catalysts are in particular low-molecular-weight organic acids, such as, for example, linear or branched C₁₋₆-carboxylic acids, or inorganic acids, such as, for example, oxidizing or nonoxidizing Brönsted acids or Lewis acids, but also inorganic solid acids, such as poly acids and heteropoly acids.

The inorganic acids are preferably chosen from the group consisting of hydrohalic acids, sulphuric acid, sulphurous acid, phosphoric acid, phosphorous acid, Lewis acids, polyphosphoric acids, polytungstic acids and mixtures thereof.

In an alternative process variant, the catalyst is a basic catalyst, in particular an organic or inorganic base.

A suitable basic catalyst is in particular a base chosen from the group consisting of hydroxides and oxides of metals of the first and second main groups, ammonia, triethylamine, trimethylamine and mixtures thereof. Alkali metal and alkaline earth metal hydroxides, alkali metal oxides and ammonia are suitable in particular.

A catalytic action already occurs, in the case of basic catalysts, in concentrations from 100 ppm (g/g), based on the starting material. In a preferred embodiment, with basic catalysis, the pH is >7. Basic catalysts can be used in any amount.

Since the reaction takes place under pressure, the possibility exists of adding gaseous bases, such as ammonia or trimethylamine, under pressure. Particularly preferably, ammonia is used as gaseous base. In a very particularly preferred embodiment, gaseous ammonia is additionally added under pressure in the presence of a basic catalyst. Ammonia is particularly advantageous as basic catalyst since ammonia can be easily removed from the product, for example under vacuum, and accordingly does not require any neutralization.

### EXAMPLES

The following examples clarify the implementation of the invention without a limitation being seen therein.

In the tables of results of Examples 1-4, the individual columns give the values for the reaction time τ in [s], for the concentration of the nitrile at different reaction times c_{CN} in [mM], for the concentration of the acid formed at different reaction times C_{COOH} in [mM], for the concentration of the amide formed at different reaction times C_{CONH2} in [mM], for the conversion of the nitrile C_{CN} in [%], for the area proportion of the acid formed A_{COOH} in [%], for the area proportion of the amide formed A_{CONH2} in [%], for the selectivity for the acid formed S_{COOH} in [%] and for the selectivity for the amide formed A_{CONH2} in [%]. Examples 5 to 8 and Comparative Examples 1 to 3 give, differing therefrom, the concentration of the amide used c_{CONH2} in [M], the concentration of the acid obtained C_{COOH} in [M] and the selectivity for pyridine S_{Py} in [%].

n.d.=not determined
Example 1: (Tables 1 and 2) Hydrolysis of 0.5% by weight of nicotinonitrile in pure water
Example 2: (Tables 3 and 4) Hydrolysis of 0.5% by weight of nicotinonitrile in 10 mM sulphuric acid
Example 3: (Tables 5 to 10) Hydrolysis of 0.5% by weight of nicotinonitrile in ammonia solutions with different concentrations
Example 4: (Tables 11 to 14) Hydrolysis of 5, 10 and 15% by weight of nicotinonitrile in conc. ammonia (14.7M, 25%)

### Example 1

### Hydrolysis of 0.5% of Nicotinonitrile in Pure Water

TABLE 1
Results at 200° C. and 25 MPa in water

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 96.9 | 0 | 0 | 0 | 0 | 0 | | |
| 50 | 94.6 | 0 | 0.85 | 2.3 | 0 | 0.9 | 0 | 38 |
| 72 | 93.6 | 0.06 | 1.33 | 3.4 | 0.1 | 1.4 | 1.8 | 40.5 |
| 101 | 92.3 | 0.13 | 1.82 | 4.7 | 0.1 | 1.9 | 2.9 | 40 |
| 206 | 87.3 | 0.14 | 3.59 | 9.9 | 0.1 | 3.7 | 1.5 | 38 |
| 269 | 84.2 | 0.15 | 4.74 | 13.0 | 0.2 | 4.9 | 1.2 | 37.5 |

TABLE 2
Results at 250° C. and 25 MPa in water

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 83.9 | 0.12 | 0 | 0 | 0 | 0 | | |
| 59 | 76.9 | 0.32 | 5.57 | 8.3 | 0.2 | 6.6 | 2.88 | 80.4 |
| 74 | 75.3 | 0.35 | 7.30 | 10.2 | 0.3 | 8.7 | 2.73 | 85.6 |
| 101 | 73.6 | 0.41 | 8.80 | 12.2 | 0.3 | 10.5 | 2.81 | 85.9 |
| 195 | 68.3 | 0.65 | 12.68 | 18.6 | 0.6 | 15.1 | 3.37 | 81.3 |
| 325 | 63.6 | 0.84 | 15.59 | 24.2 | 0.9 | 18.6 | 3.54 | 76.8 |
| 390 | 61.2 | 0.97 | 16.76 | 27.0 | 1.0 | 20.0 | 3.75 | 74.1 |

### Example 2

### Hydrolysis of 0.5% by Weight of Nicotinonitrile with Sulphuric Acid

TABLE 3
Results at 250° C. and 25 MPa in 10 mM sulphuric acid

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 50.5 | 0 | 0 | 0 | 0 | 0 | | |
| 45 | 48.7 | 0.27 | 0.87 | 3.4 | 0.5 | 1.7 | 15.50 | 50.0 |
| 72 | 47.7 | 0.87 | 1.37 | 5.5 | 1.7 | 2.7 | 31.66 | 49.5 |
| 98 | 46.9 | 1.34 | 1.73 | 7.0 | 2.7 | 3.4 | 37.89 | 48.9 |
| 180 | 44.2 | 2.76 | 2.93 | 12.5 | 5.5 | 5.8 | 43.69 | 46.5 |
| 255 | 41.8 | 3.90 | 3.93 | 17.2 | 7.7 | 7.8 | 44.95 | 45.4 |
| 379 | 39.3 | 4.71 | 4.87 | 22.2 | 9.3 | 9.6 | 42.02 | 43.5 |

TABLE 4
Results at 250° C. and 30 MPa in 10 mM sulphuric acid

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 41.1 | 0 | 0 | 0 | 0 | 0 | | |
| 47 | 39.6 | 0.3 | 0.9 | 3.7 | 0.7 | 2.1 | 17.8 | 58.3 |
| 75 | 38.5 | 0.8 | 1.4 | 6.2 | 2.0 | 3.5 | 32.0 | 55.9 |
| 100 | 37.3 | 1.4 | 2.0 | 9.1 | 3.5 | 4.9 | 37.9 | 54.1 |
| 182 | 34.6 | 3.2 | 3.2 | 15.9 | 7.7 | 7.9 | 48.4 | 49.8 |
| 278 | 32.3 | 4.6 | 4.1 | 21.4 | 11.3 | 10.0 | 52.6 | 46.8 |
| 385 | 30.1 | 5.9 | 5.0 | 26.6 | 14.4 | 12.2 | 53.9 | 45.7 |
| 883 | 22.0 | 10.4 | 8.1 | 46.5 | 25.3 | 19.7 | 54.4 | 42.4 |

### Example 3

### Hydrolysis of 0.5% by Weight of Nicotinonitrile in Ammonia Solutions with Different Concentrations

TABLE 5
Results at 250° C. and 30 MPa in 5 mM ammonia

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 43.93 | 0 | 0.00 | 0 | 0 | 0 | | |
| 56.2 | 22.04 | 1.08 | 19.92 | 49.8 | 2.5 | 45.3 | 4.94 | 91.0 |
| 97.6 | 18.24 | 2.03 | 22.93 | 58.5 | 4.6 | 52.2 | 7.89 | 89.3 |
| 182.6 | 12.29 | 3.92 | 26.96 | 72.0 | 8.9 | 61.4 | 12.39 | 85.2 |
| 286.3 | 8.01 | 6.08 | 29.10 | 81.8 | 13.8 | 66.2 | 16.94 | 81.0 |
| 421.3 | 4.76 | 9.13 | 29.04 | 89.2 | 20.8 | 66.1 | 23.30 | 74.1 |
| 549.5 | 2.85 | 11.69 | 28.03 | 93.5 | 26.6 | 63.8 | 28.47 | 68.2 |
| 712.1 | 1.35 | 15.61 | 26.15 | 96.9 | 35.5 | 59.5 | 36.67 | 61.4 |

TABLE 6
Results at 250° C. and 30 MPa in 50 mM ammonia

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 39.33 | 0 | 0 | 0 | 0 | 0 | | |
| 49 | 8.01 | 1.55 | 28.16 | 79.6 | 4.0 | 71.6 | 4.96 | 89.9 |
| 74 | 6.42 | 2.37 | 28.84 | 83.7 | 6.0 | 73.3 | 7.19 | 87.6 |
| 102.3 | 5.15 | 3.38 | 29.17 | 86.9 | 8.6 | 74.2 | 9.89 | 85.4 |
| 190.6 | 2.70 | 6.56 | 29.00 | 93.1 | 16.7 | 73.7 | 17.90 | 79.2 |
| 296.1 | 1.43 | 9.67 | 27.56 | 96.4 | 24.6 | 70.1 | 25.50 | 72.7 |
| 379.5 | 0.87 | 12.17 | 26.29 | 97.8 | 30.9 | 66.8 | 31.64 | 68.4 |
| 539.3 | 0.16 | 16.09 | 22.93 | 99.6 | 40.9 | 58.3 | 41.07 | 58.5 |

TABLE 7
Results at 250° C. and 30 MPa in 1.17M ammonia

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 35.30 | 0 | 0.57 | 0 | 0 | 0 | | |
| 45.9 | 0.25 | 4.73 | 30.25 | 99.3 | 13.4 | 84.1 | 13.51 | 84.7 |
| 73.4 | 0 | 7.40 | 27.21 | 100.0 | 21.0 | 75.5 | 20.95 | 75.5 |
| 99.8 | 0 | 9.77 | 25.08 | 100.0 | 27.7 | 69.4 | 27.67 | 69.4 |
| 171.9 | 0 | 14.71 | 20.53 | 100.0 | 41.7 | 56.6 | 41.68 | 56.6 |
| 292.0 | 0 | 18.46 | 16.58 | 100.0 | 52.3 | 45.4 | 52.29 | 45.4 |
| 446.9 | 0 | 21.34 | 13.28 | 100.0 | 60.5 | 36.0 | 60.46 | 36.0 |

TABLE 8
Results at 250° C. and 30 MPa in 5.88M ammonia

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 38.30 | 0 | 0.23 | 0 | 0 | 0 | | |
| 44.8 | 0 | 10.40 | 25.22 | 100.0 | 27.2 | 65.2 | 27.15 | 65.2 |
| 74.7 | 0 | 17.17 | 21.08 | 100.0 | 44.8 | 54.4 | 44.81 | 54.4 |
| 104.4 | 0 | 21.11 | 17.31 | 100.0 | 55.1 | 44.6 | 55.12 | 44.6 |
| 188.4 | 0 | 26.89 | 11.54 | 100.0 | 70.2 | 29.5 | 70.21 | 29.5 |
| 291.3 | 0 | 30.59 | 7.48 | 100.0 | 79.9 | 18.9 | 79.87 | 18.9 |
| 464.7 | 0 | 32.67 | 4.05 | 100.0 | 85.3 | 10.0 | 85.28 | 10.0 |
| 679.5 | 0 | 33.76 | 1.74 | 100.0 | 88.1 | 3.9 | 88.13 | 3.9 |

TABLE 9
Readings and results at 250° C. and 30 MPa in 14.7M ammonia

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 37.03 | 0 | 3.15 | 0 | 0 | 0 | | |
| 43.9 | 0 | 8.86 | 30.18 | 100 | 22.0 | 73 | 23.91 | 73 |
| 118.7 | 0 | 21.36 | 18.31 | 100 | 53.2 | 40.9 | 57.68 | 40.9 |
| 225.5 | 0 | 29.54 | 9.59 | 100 | 73.5 | 17.4 | 79.77 | 17.4 |
| 343.3 | 0 | 33.6 | 5.50 | 100 | 83.6 | 6.3 | 90.72 | 6.3 |
| 561.8 | 0 | 36.1 | 3.15 | 100 | 89.8 | 0 | 97.48 | 0 |

TABLE 10
Readings and results at 280° C. and 30 MPa in 5.88M ammonia

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 38.3 | 0 | 0 | 0 | 0 | 0 | | |
| 53.5 | 0 | 14.9 | 23.1 | 100 | 39 | 60 | 39 | 60 |
| 77.0 | 0 | 19.4 | 18.4 | 100 | 51 | 48 | 51 | 48 |
| 103.3 | 0 | 22 | 15.4 | 100 | 57 | 40 | 57 | 40 |
| 190.0 | 0 | 26.9 | 9.6 | 100 | 70 | 25 | 70 | 25 |
| 303.3 | 0 | 30.4 | 5.1 | 100 | 79 | 13 | 79 | 13 |
| 545.6 | 0 | 33.2 | 1.7 | 100 | 87 | 4 | 87 | 4 |

Amount of pyridine after 545.6 s less than 0.1%

### Example 4

### Hydrolysis of 5, 10 and 15% by Weight of Nicotinonitrile in Conc. Ammonia (14.7M, 25%)

TABLE 11
Results at 250° C. and 30 MPa in 14.7M ammonia and 5% by weight of
nicotinonitrile

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 366 | 0 | 28 | 0 | 0 | 0 | | |
| 117 | 26 | 112 | 257 | 93 | 31 | 62 | 33 | 67 |
| 238 | 0 | 200 | 193 | 100 | 55 | 45 | 55 | 45 |
| 590 | 0 | 294 | 85 | 100 | 80 | 15 | 80 | 15 |
| 801 | 0 | 314 | 61 | 100 | 86 | 9 | 86 | 9 |

TABLE 12
Results at 250° C. and 30 MPa in 14.7M ammonia and 10% by weight of
nicotinonitrile

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 791 | 0 | 57 | 0 | 0 | 0 | | |
| 47 | 211 | 57 | 576 | 73 | 7 | 66 | 10 | 89 |
| 117 | 72 | 177 | 596 | 91 | 22 | 68 | 25 | 75 |
| 223 | 20 | 294 | 513 | 97 | 37 | 58 | 38 | 59 |
| 576 | 0 | 526 | 311 | 100 | 67 | 32 | 67 | 32 |
| 817 | 0 | 586 | 225 | 100 | 74 | 21 | 74 | 21 |

TABLE 13
Results at 250° C. and 30 MPa in 14.7M ammonia and 15% by weight of
nicotinonitrile

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 1109 | 0 | 75 | 0 | 0 | 0 | | |
| 123 | 199 | 97 | 887 | 82 | 9 | 73 | 11 | 89 |
| 219 | 76 | 240 | 867 | 93 | 22 | 71 | 23 | 77 |
| 574 | 0 | 610 | 571 | 100 | 55 | 45 | 55 | 45 |
| 852 | 0 | 731 | 451 | 100 | 66 | 34 | 66 | 34 |

TABLE 14
Results at 280° C. and 30 MPa in 14.7M ammonia and 10% by weight of
nicotinonitrile

| τ | c_{CN} | c_{COOH} | c_{CONH2} | C_{CN} | A_{COOH} | A_{CONH2} | S_{COOH} | S_{CONH2} |
|---|---|---|---|---|---|---|---|---|
| 0 | 1038 | 0 | 81 | 0 | 0 | 0 | | |
| 51 | 232 | 63 | 824 | 78 | 6 | 72 | 8 | 92 |
| 106 | 119 | 158 | 842 | 89 | 15 | 73 | 17 | 83 |
| 312 | 0 | 386 | 659 | 100 | 37 | 56 | 37 | 56 |
| 505 | 0 | 542 | 554 | 100 | 52 | 46 | 52 | 46 |
| 699 | 0 | 599 | 493 | 100 | 58 | 40 | 58 | 40 |

Amount of pyridine after 699 s less than 0.1%.

In none of the reactions of Examples 1 to 4 under pressure and at temperatures of less than 280° C., nor of the following Examples 5 to 8, could any pyridine be detected in the product at the reaction end.

Examples 5 to 8 and the comparative examples were carried out in a tubular reactor with nicotinamide as starting material.

### Example 5

### Results at 150° C. and 30 MPa in 9.28M Ammonia

| τ | c_{CONH2} | c_{COOH} | C_{CONH2} |
|---|---|---|---|
| 0 | 1.056 | 0.053 | 0.0 |
| 284 | 0.993 | 0.116 | 6.0 |
| 533 | 0.948 | 0.161 | 10.3 |
| 801 | 0.926 | 0.183 | 12.3 |
| 893 | 0.894 | 0.214 | 15.3 |
| 1102 | 0.868 | 0.241 | 17.8 |
| 1385 | 0.836 | 0.272 | 20.8 |
| 2699 | 0.727 | 0.382 | 31.2 |

### Example 6

### Results at 250° C. and 30 MPa in 1.21M ammonia

| τ | c_{CONH2} | c_{COOH} | C_{CONH2} |
|---|---|---|---|
| 0 | 1.147 | 0.042 | 0.0 |
| 247 | 0.651 | 0.538 | 43.2 |
| 520 | 0.432 | 0.757 | 62.3 |
| 699 | 0.336 | 0.854 | 70.7 |
| 840 | 0.277 | 0.912 | 75.8 |
| 1021 | 0.211 | 0.978 | 81.6 |
| 1203 | 0.202 | 0.987 | 82.4 |
| 1694 | 0.133 | 1.056 | 88.4 |
| 2365 | 0.121 | 1.068 | 89.4 |

### Example 7

### Results at 200° C. and 30 MPa in 1.21M Ammonia

| τ | c_{CONH2} | c_{COOH} | C_{CONH2} |
|---|---|---|---|
| 0 | 1.148 | 0.041 | 0.0 |
| 264 | 0.968 | 0.221 | 15.7 |
| 544 | 0.843 | 0.346 | 26.5 |
| 791 | 0.759 | 0.430 | 33.9 |
| 916 | 0.715 | 0.475 | 37.7 |
| 1095 | 0.649 | 0.541 | 43.5 |
| 1330 | 0.569 | 0.621 | 50.4 |
| 1841 | 0.369 | 0.820 | 67.8 |
| 2636 | 0.341 | 0.849 | 70.3 |

### Example 8

### Results at 200° C. and 10 MPa in 0.3M Ammonia

| τ | c_{CONH2} | c_{COOH} | C_{CONH2} |
|---|---|---|---|
| 0 | 1.168 | 0.026 | 0.0 |
| 1263 | 0.568 | 0.626 | 51.3 |
| 2192 | 0.332 | 0.862 | 71.6 |
| 5017 | 0.117 | 1.078 | 90.0 |

In the comparative examples, nicotinamide was reacted in order to investigate the kinetics of the second reaction stage. The selectivity for the pyridine formed is given in mol % in comparison with the starting material and the acid formed.

### Comparative Example 1

### Results at 250° C. and 5 MPa in 0.3M Ammonia

| τ | c_{CONH2} | c_{COOH} | S_{Py} | C_{CONH2} | S_{CONH2} | S_{COOH} |
|---|---|---|---|---|---|---|
| 0 | 1.173 | 0.017 | 0.0 | 0.0 | 100.0 | 0.0 |
| 1234 | 0.555 | 0.635 | 0.0 | 52.6 | 47.4 | 52.6 |
| 2206 | 0.353 | 0.836 | 0.0 | 69.9 | 30.1 | 69.9 |
| 4960 | 0.131 | 1.046 | 1.2 | 88.9 | 11.1 | 87.7 |

### Comparative Example 2

### Results at 250° C. and 6 MPa in 0.3M Ammonia

| τ | c_{CONH2} | c_{COOH} | S_{Py} | C_{CONH2} | S_{CONH2} | S_{COOH} |
|---|---|---|---|---|---|---|
| 0 | 1.169 | 0.021 | 0.0 | 0.0 | 100.0 | 0.0 |
| 1246 | 0.569 | 0.621 | 0.1 | 51.3 | 48.7 | 51.3 |
| 2324 | 0.310 | 0.876 | 0.3 | 73.5 | 26.5 | 73.2 |
| 4999 | 0.119 | 1.057 | 1.2 | 89.8 | 10.2 | 88.6 |

### Comparative Example 3

### Results at 250° C. and 30 MPa in 0.1M Ammonia

| τ | c_{CONH2} | c_{COOH} | S_{Py} | C_{CONH2} | S_{CONH2} | S_{COOH} |
|---|---|---|---|---|---|---|
| 0 | 1.151 | 0.041 | 0 | 0 | 100.0 | 0 |
| 1691 | 0.515 | 0.677 | 0.0 | 55.3 | 44.7 | 55.3 |
| 2298 | 0.392 | 0.800 | 0.0 | 65.9 | 34.1 | 65.9 |
| 5236 | 0.102 | 1.055 | 3.1 | 91.2 | 8.8 | 88.1 |

1. Process for the hydrolysis of compounds of the formulae 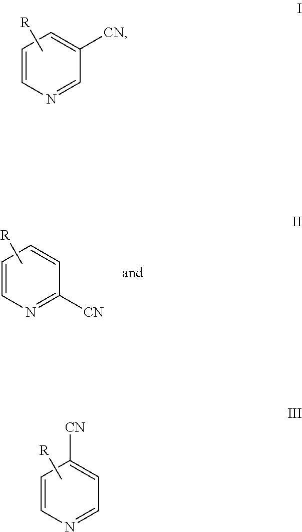
in which R is hydrogen or C₁₋₂₀-alkyl,
to give the corresponding amides and/or acids in aqueous fluids, characterized in that the reaction is carried out at a temperature of at least 100° C. and a pressure of at least 10 MPa, preferably of at least 15 MPa, particularly preferably of at least 20 MPa, if appropriate in the presence of an acid or basic catalyst.

2. Process according to claim 1, characterized in that the reaction is carried out continuously in a flow reactor with at least one reaction region.

3. Process according to claim 1, characterized in that the aqueous fluid at the beginning of the reaction comprises a nitrile of the formula I, II or III in a concentration of 0.05 to 30% by weight.

4. Process according to claim 1, characterized in that a catalyst is used in a concentration of at least 0.3 mol/l.

5. Process according to claim 1, characterized in that the catalyst is an organic or inorganic base.

6. Process according to claim 5, characterized in that the catalyst is an inorganic base chosen from the group consisting of hydroxides and oxides of metals of the first and second main groups; ammonia, triethylamine and trimethylamine; and mixtures thereof.

7. Process according to claim 1, characterized in that the basic catalyst is ammonia.

8. Process for the hydrolysis of compounds of the formulae 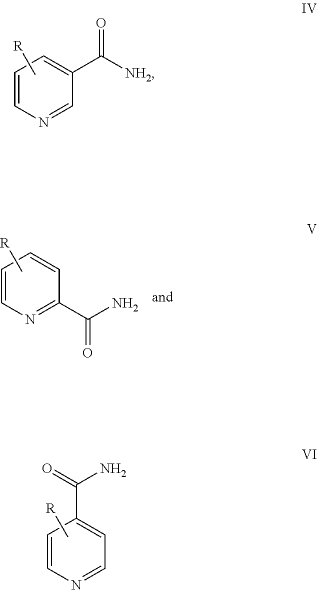
in which R is hydrogen or C₁₋₂₀-alkyl,
to give the corresponding acids in aqueous fluids, characterized in that the reaction is carried out at a temperature of at least 100° C. and a pressure of at least 10 MPa, preferably of at least 15 MPa, particularly preferably of at least 20 MPa, if appropriate in the presence of an acid or basic catalyst.

9. Process according to claim 8, characterized in that the reaction is carried out continuously in a flow reactor with at least one reaction region.

10. Process according to claim 8, characterized in that the aqueous fluid at the beginning of the reaction comprises an amide of the formula IV, V or VI in a concentration of 0.05 to 30% by weight.

11. Process according to claim 8, characterized in that a catalyst is used in an amount of 0.3 mol/1.

12. Process according to claim 8, characterized in that the catalyst is an organic or inorganic base.

13. Process according to claim 8, characterized in that the catalyst is an inorganic base chosen from the group consisting of hydroxides and oxides of metals of the first and second main groups; ammonia, triethylamine and trimethylamine; and mixtures thereof.

14. Process according to claim 8, characterized in that the catalyst is ammonia.
